(19) United States
(12) Patent Application Publication
(10) Pub. No.: US 2025/0095796 A1
(43) Pub. Date: Mar. 20, 2025

(54) METHODS OF DESIGNING POLYMERS AND POLYMERS DESIGNED THEREFROM

(71) Applicant: Toyota Research Institute, Inc., Los Altos, CA (US)

(72) Inventors: Arash Khajeh, Merced, CA (US); Ha-Kyung Kwon, New York, NY (US); Daniel Schweigert, Richmond, CA (US); Zhenze Yang, Cambridge, MA (US); Weike Ye, Sunnyvale, CA (US); Xiangyun Lei, Atlanta, GA (US)

(73) Assignees: Toyota Research Institute, Inc., Los Altos, CA (US); Toyota Jidosha Kabushiki Kaisha, Toyota-shi (JP)

(21) Appl. No.: 18/794,295

(22) Filed: Aug. 5, 2024

Related U.S. Application Data
(60) Provisional application No. 63/582,871, filed on Sep. 15, 2023.
(60) Provisional application No. 63/606,190, filed on Dec. 5, 2023.

(51) Int. Cl. G16C 20/50; G16C 20/30; G16C 20/70

(52) U.S. Cl. CPC G16C 20/50; G16C 20/30; G16C 20/70

(57) ABSTRACT

A method for designing polymers includes translating polymer representations of a training dataset and a test dataset into a format comprehensible by a generative pretraining transformer (GPT)-based model, training the GPT-based model with the translated polymer representations, generating new polymer representations, in a predefined format, using the trained GPT-based model, predicting at least one property of the generated new polymer representations using a machine learning (ML) property predictive model and selecting a first subset of the generated new polymer representations as a function of the at least one predicted property, and calculating the at least one property of the first subset of the generated new polymer representations using a molecular dynamics (MD) module.

### CROSS-REFERENCE TO RELATED APPLICATIONS

This application claims the benefit of U.S. Provisional Application No. 63/582,871, filed Sep. 15, 2023, and U.S. Provisional Application No. 63/606,190, filed Dec. 5, 2023, both of which are incorporated herein in their entirety by reference.

### TECHNICAL FIELD

The present disclosure relates generally to polymers and particularly to the inverse design of polymers using generative artificial intelligence.

### BACKGROUND

Polymers serve as functional and/or aesthetic materials for components, devices, and/or machines such as vehicle bumpers, cell phone covers, and battery electrolytes, among others. However, the development of new polymers with enhanced properties is a timely and costly process.

The present disclosure addresses issues related to the development and/or discovery of new polymers, and other issues related to polymers.

### SUMMARY

this section provides a general summary of the disclosure and is not a comprehensive disclosure of its full scope or all of its features.

In one form of the present disclosure, a method for designing polymers includes translating polymer representations of a training dataset and a test dataset into a format comprehensible by a generative pretraining transformer (GPT)-based model, training the GPT-based model with the translated polymer representations, generating new polymer representations, in a predefined format, using the trained GPT-based model, predicting at least one property of the generated new polymer representations using a machine learning (ML) property predictive model and selecting a first subset of the generated new polymer representations as a function of the at least one predicted property, and calculating the at least one property of the first subset of the generated new polymer representations using a molecular dynamics (MD) module.

In another form of the present disclosure, a system for designing polymers includes a processor and a memory communicably coupled to the processor and storing machine-readable instructions that, when executed by the processor, cause the processor to: train a generative pretraining transformer (GPT)-based model with the polymer representations, generate new polymer representations, in a predefined format, using the trained GPT-based model, predict at least one property of the generated new polymer representations using a machine learning (ML) property predictive model and selecting a first subset of the generated new polymer representations as a function of the at least one predicted property, and calculate the at least one property of the first subset of the generated new polymer representations using a molecular dynamics (MD) module.

In still another form of the present disclosure, a method for designing polymers using a machine learning system includes selecting a training dataset and a test dataset containing tokenized p-SMILES strings of known monomers of polymer electrolytes, training the GPT-based model with the tokenized p-SMILES strings, generating new polymer representations in a p-SMILES format using the trained GPT-based model, predicting at least one property of the generated new polymer representations using a machine learning (ML) predictive property model and selecting a first subset of the generated new polymer representations as a function of the at least one predicted property, and calculating the at least one property of the first subset of the generated new polymer representations using a molecular dynamics (MD) module.

### BRIEF DESCRIPTION OF THE DRAWINGS

The present teachings will become more fully understood from the detailed description and the accompanying drawings, wherein:

FIG. 1 is a block diagram illustrating an example of a machine learning system for predicting new polymers according to the teachings of the present disclosure;

FIG. 2 is graphical plot comparing the predefined metrics of a minGPT model, a 1Ddiffusion model, and a Diffusion-LM model used to unconditionally generate polymers according to the teachings of the presentation disclosure;

FIG. 3A is a graphical plot of density as a function of p-SMILES string length for a training data set and potential new polymers unconditionally generated with a minGPT model, a 1Ddiffusion model, and a Diffusion-LM model according to the teachings of the presentation disclosure;

FIG. 3B is a graphical plot of density as a function of ionic conductivity for a training data set and potential new polymers unconditionally generated with a minGPT model, a 1Ddiffusion model, and a Diffusion-LM model according to the teachings of the presentation disclosure;

FIG. 3C is a graphical plot of density as a function of transference number for a training data set and potential new polymers unconditionally generated with a minGPT model, a 1Ddiffusion model, and a Diffusion-LM model according to the teachings of the presentation disclosure;

FIG. 4A is a graphical plot of number (Count) as a function of ionic conductivity for high conductivity polymer test data (i.e., polymers in a test dataset labeled with high conductivity), low conductivity polymer test data (i.e., polymers in the test dataset labeled with low conductivity), and high conductivity oversampled test data;

FIG. 4B is a graphical plot if density as a function of ionic conductivity for a training data set and potential new polymers conditionally generated with a minGPT model, a 1Ddiffusion model, and a Diffusion-LM model according to the teachings of the presentation disclosure;

FIG. 4C is graphical plot comparing the predefined metrics of a minGPT model, a 1Ddiffusion model, and a Diffusion-LM model used to conditionally generate polymers according to the teachings of the presentation disclosure;

FIG. 4D illustrates chemical structures of five potential new polymers conditionally generated with a minGPT model, a 1Ddiffusion model, and a Diffusion-LM model according to the teachings of the presentation disclosure;

FIG. 4E illustrates the results of conditional generation of potential new polymers having ether functional groups according to the teachings of the presentation disclosure;

FIG. 5A is a graphical plot of transference number as a function of ionic conductivity for test data and the top 50 potential new polymers generated with a minGPT model according to the teachings of the presentation disclosure;

FIG. 5B is a graphical plot of ionic conductivity for the top 45 potential new polymers generated with a minGPT model according to the teachings of the presentation disclosure;

FIG. 6 shows a listing of newly discovered polymers in p-SMILES format and 2D structure format, and the corresponding ionic conductivity calculated with molecular dynamics for the newly discovered polymers; and

FIG. 7 is a flow chart for a method of designing new polymers according to the teachings of the present disclosure.

### DETAILED DESCRIPTION

The present disclosure provides new polymers, and systems and methods for designing new polymers. The new polymers exhibit enhanced properties and/or an enhanced combination of properties, with non-limiting examples of such properties including heat capacity, heat conductivity, thermal expansion, crystallinity, permeability, elastic modulus, tensile strength, resilience, refractive index, electrical conductivity, ionic conductivity, transference number, anion diffusivity, cation diffusivity, and density, among others. In some variations, the new polymers are new electrolyte polymers, i.e., polymers to be used as and/or included in a battery electrolyte. And in such variations, the systems and methods disclosed herein design new electrolyte polymers.

The systems and methods for designing new polymers include training a generative pretraining transformer (GPT) with tokenized polymer SMILES (p-SMILES) strings of known polymers (training data), and generating new polymers with the trained GPT. In some variations, the tokenized p-SMILES strings represent monomers of known polymers. In some variations, the GPT is trained with tokenized p-SMILES strings that do not include any polymer property information. In the alternative, or in addition to, the GPT is trained with tokenized p-SMILES strings that do include polymer property information, e.g., corresponding ionic conductivity values (experimental and/or calculated) for the known polymers. In variations where the GPT is trained with tokenized p-SMILES strings that do include polymer property information, the trained GPT generates p-SMILES strings and at least a portion of the generated p-SMILES strings are novel, i.e., were not included in the training data and are not known to exist. In addition, one or more properties of polymers corresponding to the novel p-SMILES strings are estimated with a machine learning (ML) model and a subset of these novel polymers are subjected to molecular dynamics (MD) simulations for calculation of one or more properties (i.e., values of the one or more properties) of the novel polymers. And in some variations, the systems and methods include a feedback loop that provides the novel polymers, with or without corresponding property values, to the training data such that additional data is available for training the GPT.

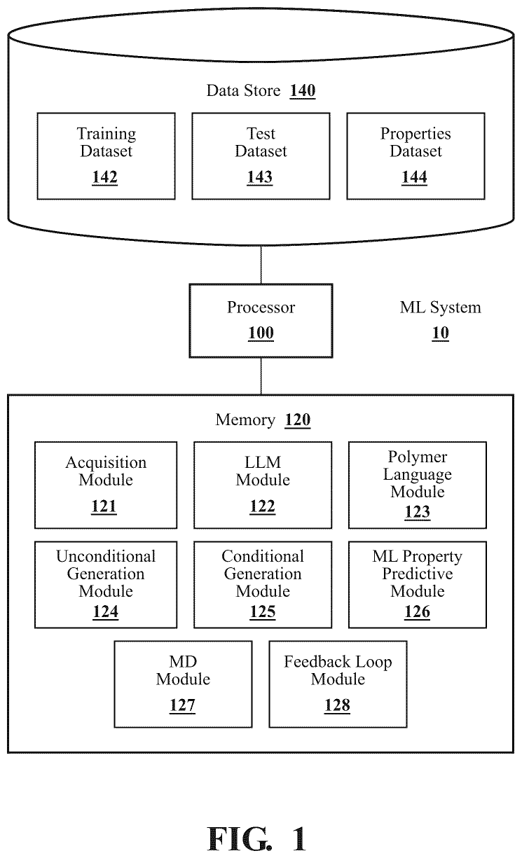

Referring to FIG. 1, a ML system 10 for predicting electrolyte polymers is illustrated. The ML system 10 is shown including one or more processors 100 (referred to herein simply as “processor 100”), a memory 120 and a data store 140 communicably coupled to the processor 100. It should be understood that the processor 100 can be part of the ML system 10, or in the alternative, the ML system 10 can access the processor 100 through a data bus or another communication path.

The memory 120 is configured to store an acquisition module 121, a large language model (LLM) module 122, a polymer language module 123, a ML property predictive module 126, a molecular dynamics (MD) module 127, and a feedback loop module 128. The memory 120 is a random-access memory (RAM), read-only memory (ROM), a hard-disk drive, a flash memory, or other suitable memory for storing the acquisition module 121, LLM module 122, polymer language module 123, ML property predictive module 126, molecular dynamics (MD) module 127, and feedback loop module 128. Also, the acquisition module 121, LLM module 122, the polymer language module 123, ML property predictive module 126, molecular dynamics (MD) module 127, and feedback loop module 128 (collectively referred to herein as “modules 121-128”) are, for example, computer-readable instructions that when executed by the processor 100 cause the processor(s) to perform the various functions disclosed herein.

In some variations the data store 140 is a database, e.g., an electronic data structure stored in the memory 120 or another data store. Also, in at least one variation the data store 140 in the form of a database is configured with routines that can be executed by the processor 100 for analyzing stored data, providing stored data, organizing stored data, and the like. Accordingly, in some variations the data store 140 stores data used by one or more of the modules 121-128. For example, and as shown in FIG. 1, in at least one variation the data store stores a training polymer dataset 142 (also referred to herein simply as “training dataset 142”), a test polymer dataset 143 (also referred to herein simply as “test dataset 143”), and a polymer properties dataset 144 (also referred to herein simply as “properties dataset 144”).

In some variations the training dataset 142 includes a listing of known polymers (i.e., chemical representations of known polymers) used to train the LLM module 122 (sometimes referred to as “ground-truth data”), the test dataset 143 includes a listing of known polymers used to test the LLM module 122, and the properties dataset 144 includes polymer properties (i.e., values of polymer properties) for at least a portion of the polymers in the training dataset 142 and/or the test dataset 143. In at least one variation, the training dataset 142 and the test dataset 143 are obtained from a single dataset of known polymers. Stated differently, a subset of known polymers is used or included in the training dataset 142 and another subset, different than the subset, is used or included in the test dataset 143. In addition, in some variations the properties dataset 144 includes polymer properties for the known polymer s in the training dataset 142 and/or the test dataset 143.

Polymer properties that can be in the properties dataset 144 include any property that is experimentally known and/or calculated for the known polymers in the training dataset 142 and/or the test dataset 143. For example, and without limitation, polymer properties in the properties dataset 144 include heat capacity, heat conductivity, thermal expansion, crystallinity, permeability, elastic modulus, tensile strength, resilience, refractive index, electrical conductivity, ionic conductivity, transference number, anion diffusivity, cation diffusivity, and density, among others.

The acquisition module 121 can include instructions that function to control the processor 100 to select a dataset including a plurality of polymers from the training dataset 142 and/or test dataset 143 and optionally corresponding polymer property values for the properties dataset 144. It should be understood that the polymer property values in the properties dataset 144 are properly tagged and/or associated with the plurality of polymers in the training dataset 142 and/or test dataset 143.

In one form of the present disclosure, the acquisition module 121 can include instructions that function to control the processor 100 to provide a dataset including a plurality of polymers from the training dataset 142 and/or test dataset 143 to the polymer language module 123. And in such a form, the polymer language module 123 can include instructions that function to control the processor to convert chemical representations of the polymers in the training dataset 142 and/or test dataset into a simplified molecular-input line-entry system (SMILES), for example a polymer SMILES (p-SMILES) format such that p-SMILES representations (also referred to herein as “p-SMILES strings”) of the polymers in the training dataset 142 and/or test dataset are comprehensible to (i.e., can be read by) the LLM module 122. It should be understood that the p-SMILES format is the standard SMILES combined with special characters “*” to encode ends of the monomer unit in a homopolymer chain (e.g., p-SMILES of PEO “*OCC*”).

In another form of the present disclosure, the plurality of polymers from the training dataset 142 and/or test dataset 143 are in the p-SMILES format and the acquisition module 121 can include instructions that function to control the processor 100 to provide a dataset from the training dataset 142 and/or test dataset 143 to the LLM module 122.

In some variations, the LLM module 122, ML property predictive module 126, MD module 127, and feedback loop module 128 can include instructions that function to control the processor 100 to perform or execute one or more of the following: unconditionally generate possible polymers via the LLM module 122, conditionally generate possible polymers via the LLM module 122, predict one or more properties of the unconditionally generated and/or conditionally generate polymers via the MPL property predictive module 126, calculate one or more properties of the unconditionally generated and/or conditionally generate polymers via the MD module 127, and provide feedback related to one or more of the unconditionally generated and/or conditionally generate polymers to the LLM module 122 via the feedback loop module 128. As used herein, the phrase “unconditionally generate” refers to generating possible polymers using a LLM module that has been trained with p-SMILES strings without any polymer property data associated therewith, and the phrase “conditionally generate” refers to generating possible polymers using a LLM module that has been trained with p-SMILES strings with polymer property data associated therewith.

Non-limiting examples of a ML model used in the ML property predictive module 126 include supervised ML models such as nearest neighbor models, Naïve Bayes models, decision tree models, linear regression models, support vector machine (SVM) models, and neural network models, among others. In at least one variation, the ML model is a graph neural network (GNN) model. And non-limiting examples of a LLM used in the LLM module 122 include generative pretrained transformer (GPT) models, and in some variations the LLM is a minimal version of a GPT model, hereafter referred to as a “minGPT” model.

Not being bound by theory, a GPT model is a model (computer code) grounded in the transformer architecture (i.e., a neural network architecture that changes an input sequence into an output sequence by learning context and tracking relationships between sequence components) that employs self-attention to dynamically weigh and focus on relevant input tokens such that long-range dependencies within a sequence are captured. In addition the minGPT model exclusively uses a decoder-based architecture and thereby emphasizes the generation aspect of its design.

In order to better embody the teachings of the present disclosure, but not limit the scope thereof in any manner, one or more examples of the ML system 10, the use of the ML system 10, methods for predicting polymers and/or the design of new polymers is provided below.

Still referring to FIG. 1, in one form of the present disclosure the ML system 10 included a previously curated dataset (training dataset) known as and referred to herein as “HTP-MD dataset” which contains 6024 different amorphous polymer electrolytes and their respective MD simulated ion transport properties as disclosed and described in the references Xie et al., “A cloud platform for sharing and automated analysis of raw data from high throughput polymer MD simulations”, APL Machine Learning, 1 (4): 046108, 11 2023, and Xie et al., “Accelerating amorphous polymer electrolyte screening by learning to reduce errors in molecular dynamics simulated properties”, Nature communications, 13 (1): 1-10, 2022, both of which are incorporated herein by reference. In addition, the LLM module 122 included a minGPT model (github.com/karpathy/minGPT) that was trained using the HTP-MD dataset.

For comparison and/or validation purposes, results (i.e., polymers) generated by the minGPT model were compared to results generated using two diffusion-based models. Particularly, a 1D denoising diffusion probabilistic model (referred to herein as the “1Ddiffusion” model) as disclosed in the reference Ho et al., “Denoising diffusion probabilistic models”, Advances in Neural Information Processing Systems 33 (NeurIPS 2020), and a diffusion language model (referred to herein as “diffusion-LM” model) as disclosed in the reference Li et al., “Diffusion-1m improves controllable text generation”, Neural Information Processing, May 22, 2022, were used to generate results using the same training dataset.

Before training of the minGPT model, the processor 100, p_SMILES strings of the 6024 different amorphous polymer electrolytes (also referred to herein as “electrolyte polymers”) in the HTP-MD dataset were further tokenized into sequential representation using the DeepChem package as disclosed in Ramsundar et al., “Deep Learning for the Life Sciences”, O'Reilly Media, 2019. https://www.amazon.com/Deep-Learning-Life-Sciences-Microscopy/dp/1492039837. Also, the tokenized p-SMILES strings were padded to a uniform length of 64, and considering the 6024 distinct repeat units of electrolyte polymers within the HTP-MD dataset, there were 20 total tokens, which included special characters that signify the start and end of a sequence as well as a padding token. Also, the HTP-MD dataset was split into a training dataset (e.g., training dataset 142) and a test dataset (e.g., test dataset 143), with 80% of the HTP-MD dataset used for training and 20% of HTP-MD dataset used for testing. And a random integer between 1 and 10 was used as an initial token for the minGPT model to start next-token (p-SMILE) prediction.

The minGPT model, the 1Ddiffusion model, and the diffusion-LM model (referred to herein collectively as “models”) were initially trained with the p-SMILES strings of the HTP-MD dataset without any property constraints in order to establish a foundation for pretraining on an unlabeled dataset that was investigated and discussed below, and to create a universal model for polymers that was not limited to one or two specific objectives. In addition, p-SMILES strings generated without any property constraints by the three models (minGPT, 1Diffusionm diffusion-LM) were classified as unconditional generated p-SMILES strings (also referred to herein as “unconditionally generated electrolyte polymers”), and the unconditionally generated electrolyte polymers were evaluated using six metrics referred to herein as novelty, uniqueness, validity, synthesizability, similarity, and diversity.

The metric “novelty” refers to the proportion of generated polymers that do not exist in the training set since the generation target was to produce polymers not in the HTP-MD dataset. The metric “uniqueness” refers to percentage of non-duplicate polymers within a generated set that allowed evaluation of whether the models could generate different polymers. The metric “validity” refers to the proportion of chemically valid p-SMILES strings generated by the models such that evaluation of the models regarding learning the correct chemical language could be performed. The metric “synthesizability” refers to the percentage of polymers considered easy to synthesize for potential experimental synthesis in the future. The metric “similarity” refers to a similarity score comparing the composition and structure of polymers in the training set with the composition and structure of polymers in the generated set, thereby indicating whether the models were learning from existing polymers. And the metric “diversity” refers to a dissimilarity score among the generated polymers, thereby evaluating whether the generated polymers were diverse so that no mode collapse issue occurred in the generation of the polymers.

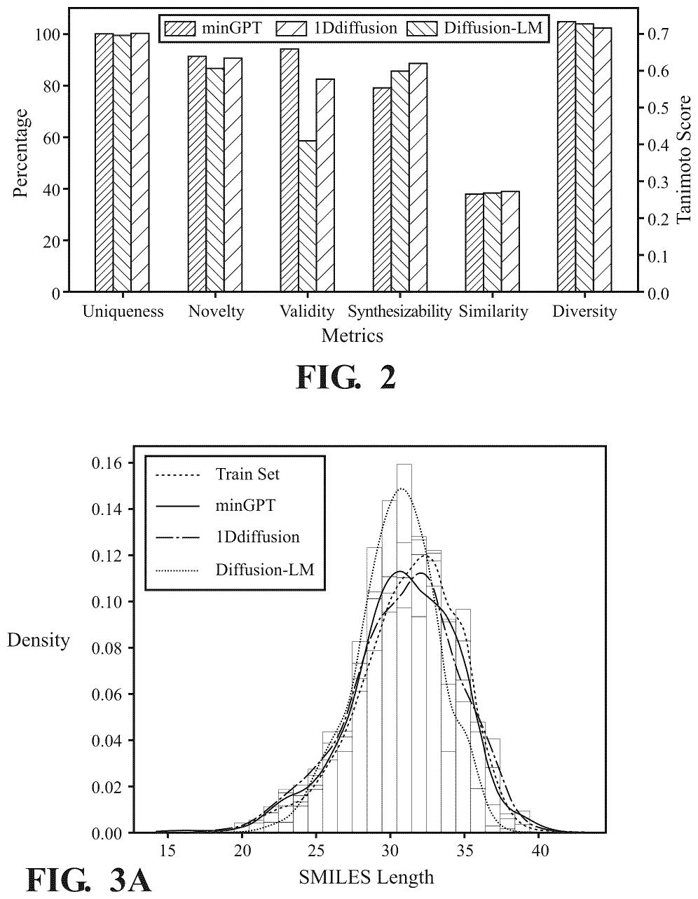

Referring to FIG. 2 performances of the minGPT, 1Ddiffusion, and diffusion-LM models as a function of the six metrics described above is shown. For both the minGPT and diffusion-LM models, high levels of novelty, uniqueness, validity, and synthesizability were observed, with most values exceeding 0.8 (80%). Accordingly, the majority of the unconditionally generated electrolyte polymers generated were novel, valid, and potentially synthesizable. In contrast, the 1Ddiffusion model underperformed compared to the other two models in producing valid electrolyte polymers.

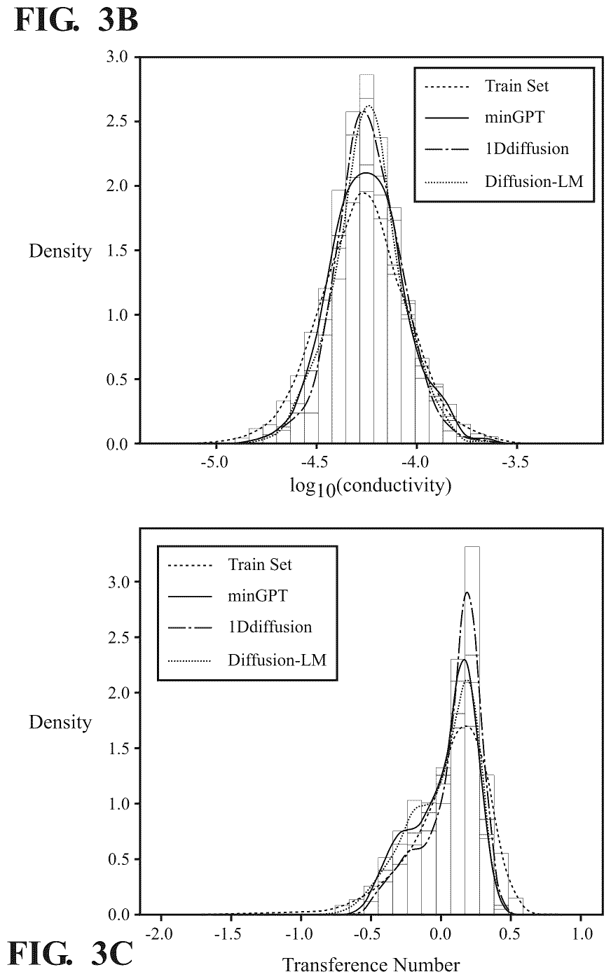

The performances of these models were also compared to each other by assessing their ability to replicate distributions of various electrolyte polymer properties, including the length of the p-SMILES string, conductivity, and transference number. Among these properties, the transference number is the fraction of the total ionic charge carried by a particular ion species in the electrolyte, and as used herein, the phrase “transference number” refers to cation transference number. Not being bound by theory, polymer electrolytes with a higher ionic conductivity and cation transference number lead to more efficient and safer battery operation. Also, calculation of the density, ionic conductivity, and transference number for the unconditionally generated electrolyte polymers was executed using the ML property predictive module 126 in the form of a graph neural network (GNN) as disclosed in Xie et al., “A cloud platform for sharing and automated analysis of raw data from high throughput polymer MD simulations”, API. Machine Learning, 1 (4): 046108, 11 2023. And with reference to FIGS. 3A-3C, the minGPT model reproduced distributions accurately for all three properties. In contrast, the other two diffusion-based models produced narrower distributions for the three properties compared to the training data, with quantitative evaluation based on the difference calculation of two distributions using Kullback-Leibler (KL) divergence.

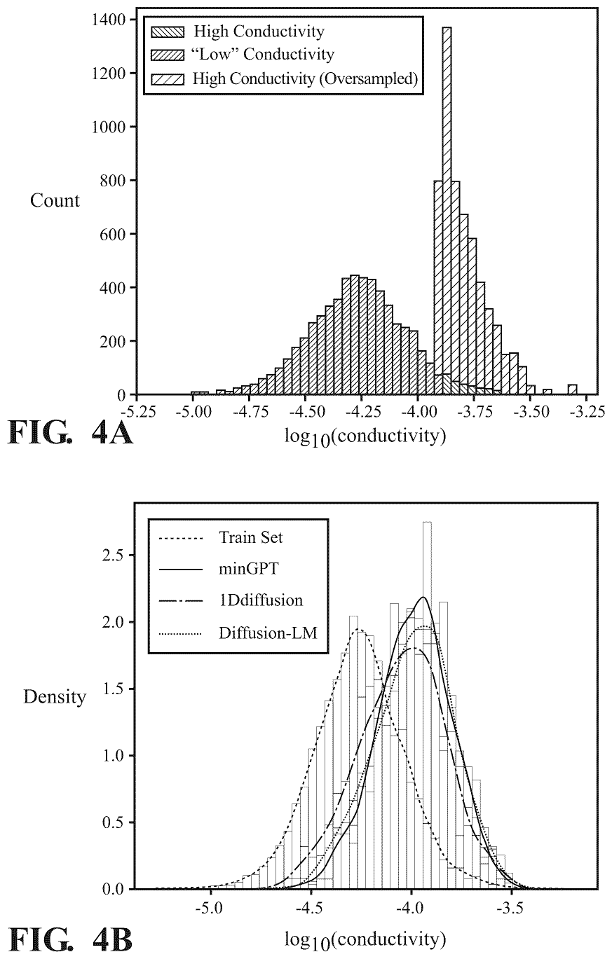

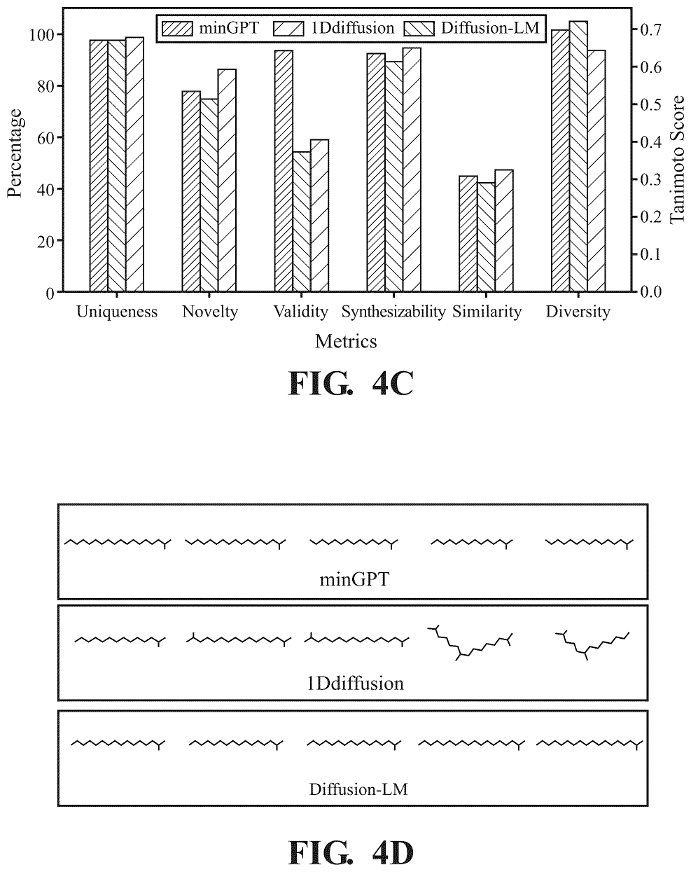

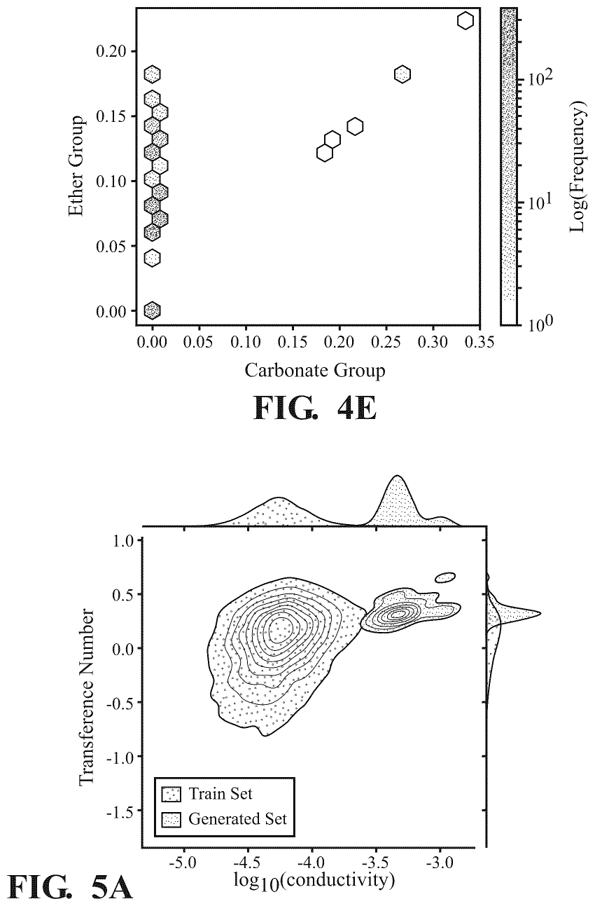

In addition to unconditional generation of possible electrolyte polymers, the system 100 was employed to conditionally generate potential electrolyte polymers with desirable properties such as high ionic conductivity (referred to herein as conditionally generated electrolyte polymers”). To achieve this objective, the polymers from the HTP-MD dataset were separated into high-conductivity and low-conductivity groups. Specifically, the top 5% of electrolyte polymers in the HTP-MD dataset were selected as the high-conductivity group, which resulted in an imbalanced dataset. To prevent training a biased model, the electrolyte polymers in the high-conductivity group were oversampled by randomly replicating the data. After oversampling, the number of electrolyte polymers in the high-conductivity group was the same as that of the low-conductivity group with the data distribution as depicted in FIG. 4A. A conductivity label was incorporated into the input sequence using special characters (“[Ag]”, “[Ac]” that were not present in any of polymer sequences (i.e., p-SMILES strings), where [Ag] represented “high conductivity” and [Ac] represented “low conductivity.” Also, the diffusion-based models were trained to generate polymer sequences with conductivity labels.

After training the three models (i.e., the minGPT, 1Ddiffusion, and Diffusion-LM models) with the high-conductivity group and the low conductivity group, the three models conditionally generated potential new electrolyte polymers and the ML property predictive module 126 was employed to predict the ionic conductivity of the conditionally generated polymers with FIG. 4B illustrating the conductivity distribution of the conditionally generated electrolyte polymers. Comparing the generated set with the training set, a distinct shift toward the high-conductivity domain for all generated sets from the three models was observed. Also, comparison of the models using the six metrics noted above was plotted (FIG. 4C), and similar to the findings from the unconditional generation (FIG. 2), the minGPT model surpassed both the 1Ddiffusion and diffusion-LM models, achieving a superior average score. And with reference to FIG. 4D, the top five candidates from the conditionally generated sets of the three models are shown, with the majority of the generated polymers possessing linear backbones and containing the “—O-CH2-CH2-” fragment within their chains. Given the high ionic conductivity of PEO-like polymers, such an observation is to be expected. Accordingly, generative models like the GPT-based model can initiate with conductivity labels and subsequently generate highly ionic conductivity polymer units.

Beyond conditioning polymer generation on conductivity, electrolyte polymers with specific functional groups were designed. Not being bound by theory, ether groups potentially enhance ion conductance in polymer electrolytes as the oxygen atoms contain negative charges that can contribute to ion diffusion process. In contrast, polymers with carbonate groups are likely to form a liquid-like phase because the carbonate group is readily cleaved, especially at high temperatures, and no longer useful as solid polymer electrolytes.

To design polymer electrolytes with specific functional groups while excluding undesired ones, two labels to the polymer sequence were added: one representing the ether group and one representing the carbonate group. More specifically, two additional input tokens were incorporated right before the p-SMILES string to guide the polymer generation process in order to ensure the inclusion or exclusion of specific target functional groups. Each input token had two options, corresponding to the presence or absence of a particular functional group in the polymers. Particularly, and in this case, the first token implied whether the polymer contained an ether group, while the second token indicated the presence of a carbonate group.

The minGPT module 122 was trained using the labeled p-SMILES strings, and during generation, the minGPT module 122 utilized two functional group input labels as starting tokens to generate complete polymer sequences. And by manipulating these two functional group input labels, polymers were deliberately generated with an ether group but without carbonate group. Particularly. and with reference to FIG. 4E, results of conditional generation with respect to the ether and carbonate functional groups is shown. And as observed from the figure, most of the conditionally generated polymers did not contain any carbonate groups, while all of the conditionally generated polymers possessed an ether group with varying numbers between instances. Accordingly, the teachings of the present disclosure provide for polymer generation conditioned with diverse design objectives such as high ionic conductivity, high transference number, presence of desired functional groups, and absence of undesired functional groups, among others.

Returning to the design or polymers with high conductivity, the minGPT module 122 conditionally generated 100,000 polymers with high-conductivity labels, and these 100,000 polymers were evaluated using the ML Property Predictive module 126. Then, the top 50 candidates were selected for further validation through MD simulations using the MD module 127. The MD simulation approach aligned with established high-throughput MD methodologies as disclosed in Xie et al., “Accelerating amorphous polymer electrolyte screening by learning to reduce errors in molecular dynamics simulated properties”, Nature communications, 13 (1): 1-10, 2022, and Khajeh et al., “Early prediction of ion transport properties in solid polymer electrolytes using machine learning and system behavior-based descriptors of molecular dynamics simulations”, Macromolecules, 56:4787-4799, 7 2023. doi: 10.1021/acs.macromol.3c00416.

The MD simulation initiated with formation of an amorphous polymer-salt system, followed by steps of system relaxation, equilibration, and a production run. Ion transport properties were subsequently determined using the cluster Nernst-Einstein method, leveraging data from the production phase. Particularly, the MD simulations were performed on polymer-Li⁺. TFSI⁻ systems within the Large Atomic Molecular Massively Parallel Simulator (LAMMPS) as disclosed in Plimpton, “Fast parallel algorithms for short-range molecular dynamics” Journal of Computational Physics, 117:1-19, 1995, thereby leveraging the Polymer Consistent Forcefield (PCFF+) as disclosed in Sun, “Force field for computation of conformational energies, structures, and vibrational frequencies of aromatic polyesters”, Journal of Computational Chemistry, 15 (7): 752-768, 1994, to describe the interactions between polymers, Li⁺ cations, and TFSI⁻ anions.

The initial system configuration was generated in the MedeA software from Materials Design, Inc., San Diego, CA, by inserting the polymer chains into a simulation box using a Monte Carlo algorithm. Fifty (50) Li+ and 50 TFSI− ions were then inserted into the simulation box in the LAMMPS molecular dynamics program from Sandia National Laboratories, with the aim of generating a system with a molality of approximately 1.50 mol/kg. The simulation procedure included an initial relaxation/equilibration phase as disclosed in Molinari et al., “Effect of salt concentration on ion clustering and transport in polymer solid electrolytes: A molecular dynamics study of peo-litfsi”, Chemistry of Materials, 30 (18): 6298-6306, 2018, and featured several annealing cycles, specifically heating and cooling, as well as compression and decompression steps to achieve densities close to theoretical values, employing sequential NVT (constant number of particles, volume, and temperature) and NPT (constant number of particles, pressure, and temperature) ensembles. This phase lasts for 5 nanoseconds (ns), ensuring adequate system relaxation was achieved at the final temperature of 353 K and atmospheric pressure.

Subsequently, a production run was conducted under NVT conditions at 353 K for another 5 ns with a time step of 2.0 femtoseconds (fs), during which the system's ionic transport properties were computed using the cluster Nernst-Einstein equation (see Lanord et al., “Correlations from ion pairing and the nernst-einstein equation”, Phys. Rev. Lett . . . 122: 136001. April 2019). And a Nosé-Hoover thermostat (see Nose, “A molecular dynamics method for simulations in the canonical ensemble”, Molecular physics, 52 (2): 255-268, 1984, and Hoover, “Canonical dynamics: Equilibrium phase-space distributions” Physical review A, 31 (3): 1695, 1985) and a temperature damping parameter of 200 fs were used in this step. An example of relaxation/equilibration and production run LAMMPS scripts are included in the PolyGen Github repository at https://github.com/TRI-AMDD/PolyGen. The simulations yielded trajectories that were analyzed to determine the ion transport properties of the he top 50 candidates using the analysis code used to compute ionic conductivity and other properties available in the HTP-MD Github repository at https://github.com/TRI-AMDD/htp_md. However, due to stability issues and limitations of the force field, only 45 out of the 50 candidates were successfully simulated.

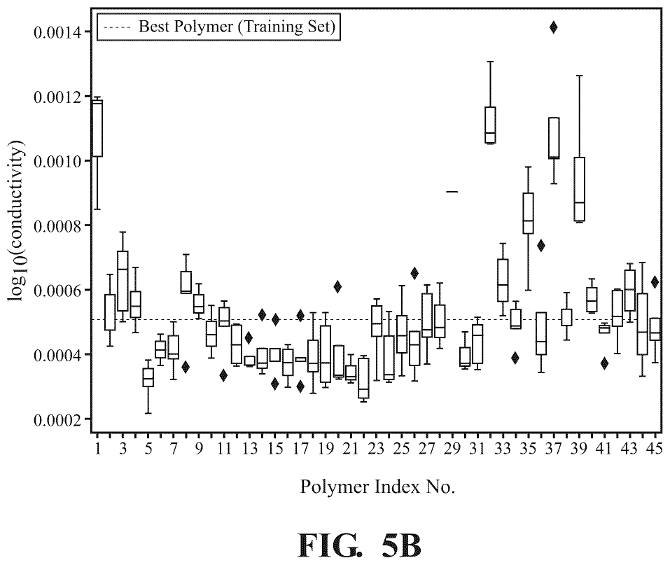

Referring to FIG. 5A, the distribution of ionic conductivity and transference number of the top 45 candidates among the 100,000 with high-conductivity labels is shown. As indicated in the figure, the ionic conductivities of the top 45 candidates are significantly higher than those in the existing training dataset. Notably, 17 of the 45 candidates surpass the highest conductivity polymer (conductivity=5.07×10-4 S/cm) in the training set as illustrated in FIG. 5B where the Polymer Index No. corresponds to the Index number and polymer p-SMILES string listed in Table 1 below. The best-performing candidate was CC(CNCCOCCOCCOC*)O* which achieved a conductivity of 1.13×10-3 S/cm, more than double that of the best polymer in the training set. The complete list of the top 45 candidates, along with their ionic conductivities and transference numbers, is shown below in Table 1. And despite not specifically targeting transference number during conditional generation, the transference numbers of the generated polymers were generally higher than those in the training set, thereby suggesting a positive correlation between ionic conductivity and transference number.

TABLE 1

| Index | p-SMILES | log₁₀(conductivity) (S/cm) | transference number |
|---|---|---|---|
| 1 | COCCN(CCO*)CCOC(F)* | −2.969125 | 0.345039 |
| 2 | CN(CCOCCOC(═O)*)CCOCCO* | −3.265667 | 0.310365 |
| 3 | O═C(*)OCCOCCN(CCOCCO*)C | −3.194681 | 0.305171 |
| 4 | CN(CCOCCO*)CCOCCOC(═O)* | −3.254057 | 0.295820 |
| 5 | O═C(*)OCCOCCOCCCCCOCCO* | −3.502602 | 0.258382 |
| 6 | O═CC(*)OCCOCCOCCCCN* | −3.384118 | 0.463982 |
| 7 | O═C(*)OCCOCCSCCOCCO* | −3.384943 | 0.191864 |
| 8 | O═C(*)OCCOCCOCCCOCCCOCO* | −3.235906 | 0.353354 |
| 9 | ON(*)CCCCSCCOCCN* | −3.253757 | 0.297232 |
| 10 | N(CCOCCOC(═O)*)CCOCCO* | −3.333095 | 0.298912 |
| 11 | O═C(*)OCCOCCNCCOCCCOCCO* | −3.314348 | 0.319658 |
| 12 | C═C(COCCOC(═O)*)COCCO* | −3.368428 | 0.266401 |
| 13 | C(CCOCCOC(═O)*)OCCOCCO* | −3.408635 | 0.303324 |
| 14 | O═C(*)OCCOCCOCCCOCCO* | −3.397825 | 0.287304 |
| 15 | O═C(*)OCCOCCCOCCOCCO* | −3.402064 | 0.345140 |
| 16 | O═C(*)OCCOCCCCOCCOCCO* | −3.432196 | 0.256873 |
| 17 | O═C(*)OCCOCCOCCCCOCCO* | −3.408268 | 0.240501 |
| 18 | O═C(*)OCCOCCOCCOCCCCCO* | −3.405185 | 0.258136 |
| 19 | O═C(*)OCCCOCCOCCOCCO* | −3.398906 | 0.320933 |
| 20 | O═C(*)OCCOCCOCCOCCCO* | −3.392791 | 0.352345 |
| 21 | O═C(*)OCCSCCOCCOCCO* | −3.461430 | 0.283546 |
| 22 | O═C(*)OCCOCCOCCSCCO* | −3.499644 | 0.239333 |
| 23 | O═C(*)OCCOCCCNCCOCCOCCO* | −3.321099 | 0.307898 |
| 24 | O═CC(*)OCCCCOCCOCCN* | −3.407873 | 0.443610 |
| 25 | O═C(*)OCCOCCNCCOCOCCO* | −3.332997 | 0.322760 |
| 26 | O═C(*)OCCOCCOCCCNCCOCCO* | −3.363917 | 0.375789 |
| 27 | NN(CCOCCOCCO*)COC(═O)* | −3.301647 | 0.312387 |
| 28 | O═CC(*)OCCSCCOCCN* | −3.295224 | 0.500443 |
| 29 | CN(CCCCO*)CCOCC* | −3.044249 | 0.345563 |
| 30 | O═C(*)OCCOCCOCCCOCCSCCO* | −3.400643 | 0.338953 |
| 31 | O═C(*)OCCOCCOCCOCCCCO* | −3.359230 | 0.244439 |
| 32 | CC(CNCCOCCOCCOC*)O* | −2.945849 | 0.336489 |
| 33 | O═C(*)OCCOCCNCCOCCOCO* | −3.203306 | 0.359395 |
| 34 | C═C(COC(═O)*)COCCOCCOCCO* | −3.309163 | 0.333118 |
| 35 | OC(*)OCCCOCCOCCN* | −3.090250 | 0.461286 |
| 36 | O═C(*)OCCOCCNCCOCCOCCO* | −3.311182 | 0.310748 |
| 37 | OOC(*)OCCO* | −2.959560 | 0.648682 |
| 38 | O═C(*)OCCOCCOCCOCCOCCCO* | −3.293195 | 0.338080 |
| 39 | CN(CCO*)CCCOCC* | −3.021205 | 0.330358 |
| 40 | O═C(*)OCCOCCSCCOCCOCCO* | −3.243046 | 0.275433 |
| 41 | O═C(*)OCCSCCOCCOCCOCCO* | −3.338027 | 0.307920 |
| 42 | O═C(*)SCCOCCOCCOCCO* | −3.284876 | 0.387148 |
| 43 | OOC(*)OCCNCCOCCO* | −3.226564 | 0.430534 |
| 44 | O═C(*)OCCOCCOCCOCCOCO* | −3.307341 | 0.345549 |
| 45 | O═C(*)OCCOCCOCCOCCOCCO* | −3.316360 | 0.304380 |

With MD simulations as a validation tool, a closed-loop framework (Feedback loop module 128) was established. Each iteration within the closed-loop framework included the following sequence of steps: conditional generation of polymers using the minGPT module 122; preliminary screening of the conditionally generated polymers using the ML property predictive module 126 to provide a subset (e.g., a first subset) of conditionally generated polymers, evaluation of the subset of conditionally generated polymers using the MID module 127 to provide another subset (e.g., a second subset) of conditionally generated polymers, and including or adding the second subset of conditionally generated polymers to the training dataset 142.

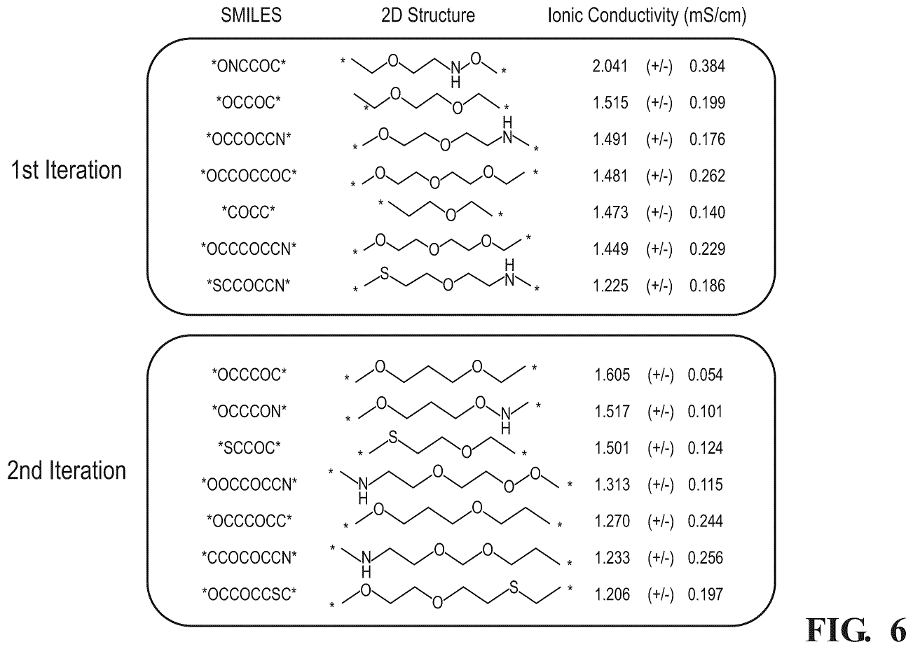

For example, and with reference to FIG. 6, a list of fourteen (14) newly discovered polymers, their corresponding two dimensional structure, and their corresponding ionic conductivity calculated using the MD module 127 is shown. Seven (7) of the polymers (i.e., *ONCCOC*, *OCCOC*, *OCCOCCN*, *OCCOCCOC*, *COCC*, *OCCCOCCN*, *SCCOCCN*) were generated after one iteration of the feedback loop module 128 providing newly discovered polymers to the training dataset 142 and seven of the polymers (i.e., *OCCCOC*, *OCCCON*, *SCCOC*, *OOCCOCCN*, *OCCCOCC*, *CCOCOCCN*, *OCCOCCSC* were generated after a second iteration of the feedback loop module 128 providing newly discovered polymers to the training dataset 142. It should be understood that each of the polymers listed in FIG. 6 exhibits an ionic conductivity greater than the ionic conductivity of PEO, and PEO is currently one of the highest conducting known dry (solid) polymers with an ionic conductivity of about 1 mS/cm at 353 K and Li TFSI molality of 1.5 mol/kg. It should also be understood that the 14 polymers in FIG. 6 that were discovered using the ML system 10 show higher ion conductivity compared to those in the original training dataset 142, and this progression indicates the potential of the ML system 10 for ongoing enhancement, leading to increasingly effective outputs via a systematic feedback approach.

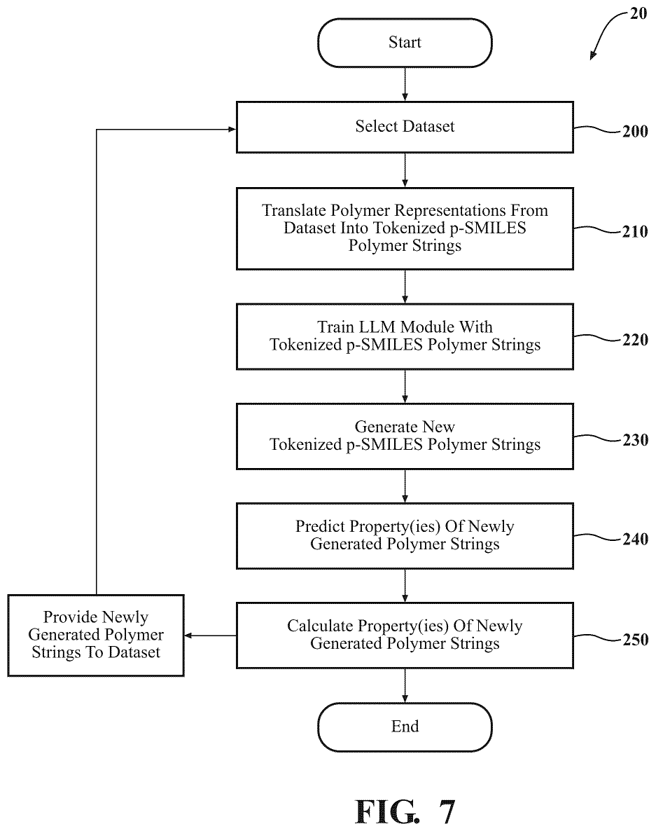

Referring now to FIG. 7, and with reference to FIG. 1, a flow chart for a method 20 of designing new polymers using the ML system 10 is shown. The method 20 includes the processor 100 selecting a training dataset 142 and a test dataset 143 of polymer representations at 200, and translating or converting the polymer representations, using the polymer language module 123, into a format that is comprehensible (i.e., can be read) by the LLM module 122 at 210. In some variations, the polymer representations in the training dataset 142 and test dataset 143 are in a predefined chemical structure format and the polymer language module 123 translates the predefined chemical structure format into a p-SMILES format (i.e., p-SMILES strings). And in at least one variation, the p-SMILES format for the polymer representations is further tokenized at the character level. In other variations, the polymer representations in the training dataset 142 and test dataset 143 are in a p-SMILES format (i.e., p-SMILES strings) and the p-SMILES strings are further tokenized as described above. And in at least one variation, the polymer representations in the training dataset 142 and test dataset 143 are in a tokenized p-SMILES format.

The method 20 proceeds to train the LLM module 122 with the tokenized p-SMILES polymer representations at 220, and the trained LLM module 122 generates new polymer representations in the p-SMILES format at 230. In some variations, the tokenized p-SMILES polymer representations do not include any property values (tokens), and in such variations the trained LLM module 122 unconditionally generates new polymer representations in the p-SMILES format at 230. In other variations, the tokenized p-SMILES polymer representations do include corresponding property values (tokens), and in such variations the trained LLM module 122 conditionally generates new polymer representations in the p-SMILES format at 230.

After the new polymer representations in the p-SMILES format have been generated, the method 20 proceeds to 240 where one or more properties of the newly generated polymer representations are predicted (estimated) with the ML property predictive module 126. And at 250, the method 20 calculates one or more properties on at least a subset of the newly generated polymer representations using the MD module 127. For example, in some variations a subset of the newly generated polymer representations with desired values (e.g., top 10%) of the one or more properties predicted by the ML property predictive module 126 are selected for MD calculations.

In some variations, the method 20 includes a feedback loop 260 that provides newly generated polymer representations to the training dataset 142 or the test dataset 143. For example, newly generated polymer representations with associated MD calculated properties can be provided to and included in the training dataset 142 or the test dataset 143 for continued training of the LLM module 122.

Based on the teachings of the present disclosure, it should be understood that a GPT module/model provides for enhanced discovery of new polymers. Particularly, GPT module/models as disclosed herein generate currently unknown polymer representations. And in combination with a ML property predictive model/module, thousands, tens of thousands, and hundreds of thousands can be filtered to provide a subset of unknown polymer representations for MD property simulations such that a significant decrease in time and cost is needed for new polymer exploration. Stated differently, in some variations a GPT module/model as disclosed herein, in combination with a ML property predictive model/module and/or a MD model/module, provides for enhanced polymer discovery not found in the prior art or known to those skilled in the art at the present time.

The preceding description is merely illustrative in nature and is in no way intended to limit the disclosure, its application, or uses. Work of the presently named inventors, to the extent it may be described in the background section, as well as aspects of the description that may not otherwise qualify as prior art at the time of filing, are neither expressly nor impliedly admitted as prior art against the present technology.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple variations or forms having stated features is not intended to exclude other variations or forms having additional features, or other variations or forms incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/−10% of the measured value, while in other variations such known commercial and/or experimental measurement tolerances are +/−5% of the measured value, while in still other variations such known commercial and/or experimental measurement tolerances are +/−2.5% of the measured value. And in at least one variation, such known commercial and/or experimental measurement tolerances are +/−1% of the measured value.

The flowcharts and block diagrams in the figures illustrate the architecture, functionality, and operation of possible implementations of systems, methods, and computer program products according to various embodiments. In this regard, a block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a ROM, an EPROM or flash memory, a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an ASIC, a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, radio frequency (RF), etc., or any suitable combination of the foregoing. Computer program code for carrying out operations for aspects of the present arrangements may be written in any combination of one or more programming languages, including an object-oriented programming language such as Java™, Smalltalk, C++, Python, or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one variation, or various variations means that a particular feature, structure, or characteristic described in connection with a form or variation, or particular system is included in at least one variation or form. The appearances of the phrase “in one variation” (or variations thereof) are not necessarily referring to the same variation or form. It should also be understood that the various method steps discussed herein do not have to be carried out in the same order as depicted, and not each method step is required in each variation or form.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. A method for designing polymers using a machine learning system, comprising:
translating polymer representations of a training dataset and a test dataset into a format comprehensible by a generative pretraining transformer (GPT)-based model;
training the GPT-based model with the translated polymer representations;
generating new polymer representations, in a predefined format, using the trained GPT-based model;
predicting at least one property of the generated new polymer representations using a machine learning (ML) property predictive model and selecting a first subset of the generated new polymer representations as a function of the at least one predicted property; and
calculating the at least one property of the first subset of the generated new polymer representations using a molecular dynamics (MD) module.

2. The method according to claim 1 further comprising tokenizing the translated polymer representations before training the GPT-based model.

3. The method according to claim 2, wherein the training dataset and the test dataset comprise representations of known monomers of polymer electrolytes.

4. The method according to claim 3, wherein the training dataset and the test dataset further comprise corresponding property values for the known monomers.

5. The method according to claim 4, wherein the corresponding property values are selected from the group consisting of ionic conductivity values, transference number values, and density values.

6. The method according to claim 1, wherein the translated polymer representations are tokenized p-SMILES strings.

7. The method according to claim 6, wherein the new polymer representations are p-SMILES strings generated by the trained GPT-based model.

8. The method according to claim 7, wherein the generated p-SMILES strings comprise one or more selected from the group consisting of *ONCCOC*, *OCCOC*, *OCCOCCN*, *OCCOCCOC*, *COCC*, *OCCCOCCN*, *SCCOCCN*, *OCCCOC*, *OCCCON*, *SCCOC*, *OOCCOCCN*, *OCCCOCC*, *CCOCOCCN*, and *OCCOCCSC *.

9. The method according to claim 1 further comprising selecting and providing a second subset of the generated new polymer representations to the training dataset or the test dataset such that training of the GPT-based model includes the second subset of the generated new polymer representations.

10. The method according to claim 9, wherein the second subset of the generated new polymer representations are selected as a function of the at least one property of the first subset of the generated new polymer representations calculated using a molecular dynamics (MD) module.

11. A system for designing polymers, the system comprising:
a processor and a memory communicably coupled to the processor and storing machine-readable instructions that, when executed by the processor, cause the processor to:
train a generative pretraining transformer (GPT)-based model with polymer representations;
generate new polymer representations, in a predefined format, using the trained GPT-based model;
predict at least one property of the generated new polymer representations using a machine learning (ML) property predictive model and selecting a first subset of the generated new polymer representations as a function of the at least one predicted property; and
calculate the at least one property of the first subset of the generated new polymer representations using a molecular dynamics (MD) module.

12. The system according to claim 11, wherein the memory communicably coupled to the processor and storing machine-readable instructions that, when executed by the processor, further cause the processor to train the GPT-based model with tokenized polymer representations.

13. The system according to claim 12, wherein the training dataset and the test dataset comprise representations of known monomers of polymer electrolytes.

14. The system according to claim 13, wherein the training dataset and the test dataset further comprise corresponding property values for the known monomers.

15. The system according to claim 14, wherein the corresponding property values are selected from the group consisting of ionic conductivity values, transference number values, and density values.

16. The system according to claim 11, wherein the polymer representations in the training dataset and the test dataset are in a p-SMILES format that is comprehensible by the GPT-based model.

17. The system according to claim 16 further comprising tokenizing the p-SMILES format of the polymer representations before training the GPT-based model.

18. The system according to claim 17, wherein the new polymer representations are one or more p-SMILES selected from the group consisting of *ONCCOC*, *OCCOC*, *OCCOCCN*, *OCCOCCOC*, *COCC*, *OCCCOCCN*, *SCCOCCN*, *OCCCOC*, *OCCCON*, *SCCOC*, *OOCCOCCN*, *OCCCOCC*, *CCOCOCCN*, and *OCCOCCSC *.

19. A method for designing polymers using a machine learning system, comprising:
selecting a training dataset and a test dataset containing tokenized p-SMILES strings of known monomers of polymer electrolytes;
training the GPT-based model with the tokenized p-SMILES strings;
generating new polymer representations in a p-SMILES format using the trained GPT-based model;
predicting at least one property of the generated new polymer representations using a machine learning (ML) predictive property model and selecting a first subset of the generated new polymer representations as a function of the at least one predicted property; and
calculating the at least one property of the first subset of the generated new polymer representations using a molecular dynamics (MD) module.

20. The method according to claim 19 further comprising selecting and providing a second subset of the generated new polymer representations to the training dataset or the test dataset such that training of the GPT-based model includes the second subset of the generated new polymer representations, wherein the second subset of the generated new polymer representations are selected as a function of the at least one property of the first subset of the generated new polymer representations calculated using a molecular dynamics (MD) module.
